(19) United States
(12) Patent Application Publication
(10) Pub. No.: US 2019/0151454 A1
(43) Pub. Date: May 23, 2019

(54) MOLECULAR ASSEMBLY USING AMPHIPATHIC BLOCK POLYMER, AND SUBSTANCE-CONVEYANCE CARRIER USING SAME

(71) Applicant: SHIMADZU CORPORATION, Kyoto-shi (JP)

(72) Inventors: Eri Matsutani, Kyoto-shi (JP); Eiichi Ozeki, Kyoto-shi (JP); Takashi Kawabe, Kyoto-shi (JP)

(21) Appl. No.: 16/258,554

(22) Filed: Jan. 26, 2019

Related U.S. Application Data
(62) Division of application No. 14/898,076, filed on Dec. 11, 2015.

(30) Foreign Application Priority Data
Jun. 12, 2013 (JP) 2013-124072

(51) Int. Cl. A61K 47/34; C08G 63/685; C08L 67/04; A61K 49/00

(52) U.S. Cl. CPC A61K 47/34; A61K 49/0002; C08L 67/04; C08G 63/6852

(57) ABSTRACT

Provided is a molecular assembly having any nano-sized particle diameter depending on its intended use and application, in various fields such as pharmaceutical drugs, agricultural, chemicals, cosmetics, food products, and electronics. Furthermore, provided is a nano-carrier for delivering various substances using the molecular assembly having any nano-sized particle diameter. A molecular assembly comprising: an amphiphilic block polymer A1 comprising a hydrophilic block having a sarcosine unit and a hydrophobic block having a lactic acid unit; and an amorphous hydrophobic polymer A2 having an aliphatic hydroxy acid unit, wherein a number of aliphatic hydroxy acid units contained in the amorphous hydrophobic polymer A2 exceeds twice a number of lactic acid units contained in the hydrophobic block of the amphiphilic block polymer A1.

### CROSS-REFERENCE TO RELATED APPLICATIONS

This application is a Divisional Application of patent application Ser. No. 14/898,076, filed on Dec. 11, 2015, which is a 371 application of Application Serial No. PCT/JP2014/065420, t filed on Jun. 11, 2014, which is based on Japanese Patent Application No. 2013-124072, filed on Jun. 12, 2013, the entire contents of which are hereby incorporated by reference.

### TECHNICAL FIELD

The present invention belongs to the fields of supramolecular chemistry, collaborative region, of medicine, engineering, and pharmacy, and nanomedicine. The present invention relates to a fine particle having a small particle diameter for use in, for example, pharmaceutical drugs, agricultural chemicals, cosmetics, food products, and electronics (e.g., battery materials), for example, a molecular assembly having a nano-level particle diameter. The nanomolecular assembly according to the present invention can be used as a nano-carrier for delivering various substances.

More specifically, the present invention relates to a molecular assembly using an amphiphilic block polymer, and a nano-carrier using the molecular assembly for use in delivering various substances. The size of the molecular assembly is controlled depending on its application, and purpose. More particularly, the present invention relates to a molecular assembly using an amphiphilic block polymer, and a nano-carrier using the molecular assembly for use in delivering a drug or a labeling agent. The nano-carrier for drug delivery can be used in a drug delivery system (DDS), and the nano-carrier for labeling agent, delivery can be used as a molecular probe for molecular imaging.

### BACKGROUND ART

As described in JP 2005-172822 A, there has been a growing interest in nanotechnology in recent years, and novel functional materials have been developed by taking advantage of properties unique to nano-sized substances. Such novel functional materials can be applied to a wide range of fields such as energy, electronics, and medicine and pharmacy. Nanotechnology has been attracting attention in, particularly, detection of substances in biological samples and in-vivo imaging. Particularly, in the field of medicine and pharmacy, for example, liposome that is a nanoparticle composed of phospholipid is used as a carrier in a drug delivery system (DDS).

In the field of medicine and pharmacy, as described in JP 2005-220045 A, it is desired that changes in the form and function of organs or tissues caused by diseases in a living body are speedily and accurately detected by a simple method at the early stage of the diseases in the diagnosis and treatment of the diseases. Particularly, in order to early diagnose and treat cancer, it is essentially necessary to determine a small lesion site and to determine the size of the lesion site at an early stage in carcinogenesis. Examples of a method for early diagnosis include endoscopic biopsy and diagnostic imaging such as radiography, MRI, and ultrasonography. However, when a radioactive indicator is used, the lifetime of the indicator is limited due to its half-life. Further, a diagnostic apparatus is also very expensive.

On the other hand, diagnostic imaging using a fluorescent indicator or a near-infrared indicator is also known. In the case of such a diagnostic method, the lifetime of an indicator itself is not greatly limited, and a measuring apparatus for diagnosis is not very expensive as compared to the apparatus using radiative rays. Further, diagnosis using light is non-invasive to a living body.

For example, autofluorescence observation via endoscope is practically used, which utilizes the fact that the autofluorescence of tumor cells is weaker than that of normal cells (excitation at 450 nm, fluorescence emission at 520 nm). When small animals are used, cancer diagnostic imaging using chemiluminescence is also performed. Chemiluminescence is a phenomenon in which a luminescent substrate (luciferin) is oxidized by the action of an enzyme (luciferase) to an unstable peroxide, and then light is emitted in the process of decomposition of the peroxide.

Further, near-infrared fluorescence imaging has also been attracting attention, which is a method for imaging a tumor site by accumulating a near-infrared fluorescent dye in the tumor site. In this method, a compound having the property of emitting fluorescence in the near-infrared region by irradiation with excitation light is administered to a living body as a contrast agent. Then, the living body is externally irradiated with excitation light having a near-infrared wavelength to detect fluorescence emitted from the fluorescent contrast agent accumulated in a tumor site and to determine a lesion site. As such a contrast agent, a nanoparticle has been reported, such as liposome having an indocyanine green derivative encapsulated therein (see JP 2005-220045 A).

On the other hand, peptide-type nanoparticles having higher biological compatibility are also known. For example, JP 2008-024816 A and US 2008/0019908 A disclose a peptide-type nanoparticle using an amphiphilic block polymer having methyl polyglutamate as a hydrophobic block. These documents describe that the particle diameter of the nanoparticle can be controlled by changing the chain length of the amphophilic block polymer. Further, these documents describe that accumulation of the nanoparticles in cancer tissue was observed.

Further, Chemistry Letters, vol. 36, no. 10, 2007, p. 1220-1221 describes that an amphophilic block polymer composed of a polylactic acid chain and a polysarcoaine chain was synthesized, and a molecular assembly with a particle diameter of 20 to 200 nm having applicability to a nano-carrier in DDS was prepared by self-assembly of the amphiphilic block polymer.

WO 2009/148121 A (US 2011/0104056 A) and Biomaterials, 2009, vol. 30, p. 5156-5160 disclose that a linear amphiphilic block polymer having a polylactic acid chain as a hydrophobic block and a polysarcosine chain as a hydrophilic block self-assembles in an aqueous solution to form a polymeric micelle (lactosome). The particle diameter of the lactosome disclosed in paragraph [0127] in WO 2009/148121 A is 10 nm to 500 nm, but the particle diameter of the lactosome actually demonstrated is only 30 nm to 130 nm disclosed in paragraph [0251]. It is known that the lactosome exhibits high retentivity in blood, and the amount of the lactosome accumulated in the liver is significantly reduced as compared to a polymeric micelle that has been already developed. This lactosome utilizes the property that a nanoparticle with a particle diameter of several tens of nanometers to several hundreds of nanometers retained in blood is likely to be accumulated in cancer (Enhanced Permeation and Retention (SPR) effect), and therefore can be applied as a nano-carrier for cancer site-targeting molecular imaging or drug delivery.

Cells grow faster in cancer tissue than in normal tissue, and therefore formation of new blood vessels is induced in cancer tissue in order to obtain oxygen and energy required for cell growth. It is known that the new blood vessels are brittle, and therefore relatively large molecules also leak from the blood vessels Further, the substance excretory system of cancer tissue is undeveloped, and therefore molecules leaking from the blood vessels are accumulated in cancer tissue for a certain period of time. This phenomenon is known as EPE effect.

WO 2012/176885 A discloses that a branched amphophilic block polymer having a branched hydrophilic block containing sarcosine and a hydrophobic block having polylactic acid self-assembles in an aqueous solution to form a polymeric micelle (lactosome) having a particle diameter of 10 to 50 nm.

JP 2009-09678 A discloses a water-dispersible nanoparticle comprising a blood circulation promoter and a biodegradable polymer (claim 1, [001]), and also discloses that the nanoparticle has a hydrophobic blood circulation promoter encapsulated therein ([0017]), the biodegradable polymer is a protein (clam 7), and the average particle size of the nanoparticles is usually 1 to 1,000 nm, preferably 10 to 1,000 nm, more preferably 10 to 500 nm, particularly preferably 15 to 400 nm ([0028]). Further, JP 2009-096787 A discloses that the blood circulation promoter is a cosmetic component, a functional food product component, a quasi-drug component, or a pharmaceutical drug component (claim 5). Further, JP 2009-09678 A discloses a drug delivery agent comprising the nanoparticle (claim 13), and also discloses that the drug delivery agent is used as a transdermal agent, a topical therapeutic agent, an oral therapeutic agent, an intradermal injection, a subcutaneous injection, an intramuscular injection, an intravenous injection, a cosmetic product, a quasi-drug, a functional food product, or a supplement (claim 14).

### PRIOR ART DOCUMENTS

### Patent Documents

Patent Document 1: JP 2005-172522 A

Patent Document 2: JP 2005-220045 A

Patent Document 3: JP 2008-024816 A

Patent Document 4: US 2008/0010908 A

Patent Document 5: WO 20097148121

Patent Document 6: US 2011/0104056 A

Patent Document 7: WO 2012/176885 A

Patent Document 8: JP 2009-096787 A

### NON-PATENT DOCUMENTS

Non-Patent Document 1: Chemistry Letters, 2007, Vol. 36, No. 10, p. 1220-1221

Non-Patent Document: 2: Biomaterials, 2009, vol. 30, p. 5156-5160

### SUMMARY OF THE INVENTION

### Problems to be Solved by the Invention

Endoscopic biopsy and diagnostic imaging such as radiography, MRI, and ultrasonography hate their respective excellent advantages, but are invasive methods imposing psychological pressure, pain or suffering, or radiation exposure on subjects.

On the other hand, as a non-invasive method, cancer diagnostic imaging using fluorescence or chemiluminescence is known. However, particularly, a method using chemiluminescence requires genetic modification, and therefore cannot be applied to humans from the viewpoint of safety.

The liposome using near-infrared light described in JP 2005-220045 A is recognized by immune system cells, such as macrophages, in blood and eliminated. Therefore, the liposome is captured by, for example, the reticuloendothelial system (RES) of the liver and spleen where a large number of macrophage-like cells are present, and is therefore poor in retentivity in blood. Further, such liposome is limited in the composition of its hydrophobic part, and therefore also has a problem in that the control of its particle size is limited.

The nanoparticle described in JP 2008-024816 A uses a peptide-type amphiphilic block polymer (peptosome). Unlike the case of liposome, at the time of production of the nanoparticles, the peptide-type amphiphilic block polymer is not dissolved in a low-boiling point solvent such as chloroform. Therefore, the nanoparticles need to be produced by a method in which the peptide-type amphiphilic block polymer is dissolved in, for example, trifluoroethanol (TFE) and then dispersed in water (i.e., by an injection method). However, TFE itself is toxic, and therefore TFE used in the injection method needs to be strictly removed by gel filtration in order to administer the nanoparticles prepared by the injection method to a living body.

Further, JP 2008-024816 A describes that this peptide-type nanoparticle is accumulated in cancer tissue by EPR (enhanced permeability and retention) effect. however, this evaluation was made based on fluorescent observation of only cancer tissue and its vicinity. For example, although not described in JP 2008-024816 A, when a mouse is observed from its ventral side, accumulation of a drug is observed also in living tissue, such as the liver or spleen, other than cancer. Therefore, when the peptide-type nanoparticle is used in fluorescent imaging, imaging of cancer tissue around the above tissue is difficult. Further, when the peptide-type nanoparticle is used in DDS, the rate of delivery of a drug to a diseased site is low.

Further, JP 2008-024816 A describes that the particle diameter of the nanoparticle can be controlled by changing the chain length of the amphiphilic block polymer. However, in fact, JP 2008-024816 A only demonstrates that two kinds of nanoparticles different in particle diameter from each other can be obtained from two kinds of amphiphilic block polymers that are the same in block chain components (structural units) but are different In chain length from each other, and that some kinds of nanoparticles different in particle diameter from each other can be obtained from some kinds of amphiphilic block polymers that are different in both block chain components (structural units) and chain length from each other. That is, JP 2008-024816 A neither discloses nor suggests the correspondence relationship between the physical amount of the amphiphilic block polymer and the particle diameter of the nanoparticle. Therefore, the particle diameter cannot be continuously controlled by the invention described in JP 2008-024816 A.

Chemistry Letters, vol. 36, no. 10, 2007, p. 1220-1221 suggests that a molecular assembly containing a polylactic acid chain is applicable to a nano-carrier in DDS. However, there is no description about the administration of the molecular assembly to a living body, and therefore, of course, there is no description about the dynamic behavior of the molecular assembly in a living body. Further, as in the case of JP 2008-024816 A, there is no description about the continuous control of the particle diameter.

As described above, WO 2009/148121 A discloses that the particle diameter of the lactosome is 10 nm to 500 nm in paragraph [0127], but the particle diameter of the lactosome actually demonstrated is only 30 nm to 130 nm disclosed in paragraph [0251].

In various fields such as pharmaceutical drugs, agricultural chemicals, cosmetics, food products, and electronics (e.g., battery materials), various nanoparticles different in particle diameter are required depending on their intended use and application.

It is therefore an object of the present invention to provide, in various fields such as pharmaceutical drugs, agricultural chemicals, cosmetics, food products, and electronics (e.g., battery materials), a molecular assembly having any nano-sized particle diameter depending on its intended use and application. It is also an object of the present invention to provide, in the shore various fields, a nano-carrier for delivering various substances using the molecular assembly having any nano-sized particle diameter depending on its intended use and application.

More particularly, it is an object of the present invention to provide a molecular assembly that is highly safe for a living body and is easy in its particle diameter control and preparation. Further, it is also an object of the present invention to provide a nano-carrier using the molecular assembly for use in delivering a drug or a labeling agent.

### Means for Solving the Problems

The present inventors have intensively studied, and as a result, have found that the above objects of the present invention can be achieved by forming a molecular assembly from a polysarcosine/polylactic acid-based amphiphilic block polymer and a polyaliphatic hydroxy acid-based amorphous hydrophobic polymer so that the number of aliphatic hydroxy acid units in the amorphous hydrophobic polymer exceeds twice the number of lactic acid units in the amphiphilic block polymer, which has led to the completion of the present invention.

The present invention includes the following.
(1) A molecular assembly comprising:

an amphiphilic block polymer A1 comprising a hydrophilic block having a sarcosine unit and a hydrophobic block having a lactic acid unit; and

an amorphous hydrophobic polymer A2 having an aliphatic hydroxy acid unit,

wherein a number of aliphatic hydroxy acid units (U_{A2}) contained in the amorphous hydrophobic polymer A2 exceeds twice a number of lactic acid units (U_{A1}) contained in the hydrophobic block, of the amphiphilic block, polymer A1 (U_{A2}>2·U_{A1}).

Here, when the number of aliphatic hydroxy acid units contained in the amorphous hydrophobic polymer A2 is represented as U_{A2}, and the number of lactic acid units contained in the hydrophobic block of the amphiphilic block polymer A1 is represented as U_{A1}, U_{A2}>2·U_{A1}. The number of structural units, that is, the degree of polymerization refers to the average degree of polymerization.

The term “molecular assembly” basically refers to a structure formed by aggregation or self-assembling orientation of molecules of the amphiphilic block polymer. A molecular assembly comprising the amphiphilic block polymer A1 containing a hydrophobic block chain having a lactic acid unit as a basic unit it sometimes referred to as “lactosome”. The term “sarcosine” refers to N-methylglycine.

The property, “hydrophilicity” of the hydrophilic block chain of the amphiphilic block polymer A1 means that the hydrophilic block chain is relatively more hydrophilic than the hydrophobic block, chain having a lactic acid unit. The property, “hydrophobicity” of the hydrophobic block chain means that the hydrophobic block chain is relatively more hydrophobic than the hydrophilic block chain having a sarcosine unit. The property, “hydrophobicity” of the hydrophobic polymer A2 means that the hydrophobic polymer A2 is relatively more hydrophobic than the hydrophilic block chain of the amphiphilic block polymer A1.
(2) The molecular assembly according to (1), wherein the hydrophilic block contains 2 to 300 sarcosine units.
(3) The molecular assembly according to (1) or (2), wherein the hydrophobic block, contains 5 to 400 lactic acid units.
(4) The molecular assembly according to any one of (1) to (3), wherein the amorphous hydrophobic polymer A2 has, as the aliphatic hydroxy acid unit, at least one selected from the group consisting of a lactic acid unit and a glycolic acid unit.
(5) The molecular assembly according to any one of (1) to (4), wherein the amorphous hydrophobic polymer A2 contains 35 or more aliphatic hydroxy acid units.
(6) The molecular assembly according to any one of (1 ) to (5), wherein the amorphous hydrophobic polymer A2 contains 200 or more aliphatic hydroxy acid units.
(7) The molecular assembly according to any one of (1) to (6), wherein a molar ratio A2/A1 of the amorphous hydrophobic polymer A2 to the amphiphilic block polymer A1 is in the range of 0.1/1 to 10/1.
(8) The molecular assembly according to any one of (1) to (7), which has a particle diameter of 10 to 1,000 nm.

The term “particle diameter” refers to a particle diameter occurring most frequently in particle size distribution, that is, a mode particle diameter.
(9) The molecular assembly according to any one of (1) to (8), which is obtained by a preparation method comprising the stops of:

preparing a solution, in a container, containing the amphiphilic block polymer A1 and the amorphous hydrophobic polymer A2 in an organic solvent;

removing the organic solvent from the solution to obtain a film comprising the amphiphilic block polymer A1 and the amorphous hydrophobic polymer A2 on an inner wall of the container; and

adding water or an aqueous solution into the container to convert the film into a particulate molecular assembly, thereby obtaining a dispersion liquid of the molecular assembly.
(10) The molecular assembly according to any one of (1) to ( 8), which is obtained by a preparation, method comprising the steps of:

preparing a solution, in a container, containing the amphiphilic block polymer A1 and the amorphous hydrophobic polymer in an organic solvent;

dispersing the solution into water or an aqueous solution; and

removing the organic solvent.
(11) A nano-carrier for delivering a substance, comprising the molecular assembly according to any one of (1) to (10).
(2-1) A method for controlling a particle diameter of a molecular assembly comprising; an amphiphilic block polymer A1 comprising a hydrophilic block having a sarcosine unit and a hydrophobic block having a lactic acid unit; and an amorphous hydrophobic polymer A2 having an aliphatic hydroxy acid unit,

wherein a number of aliphatic hydroxy acid units (U_{A2}) contained in the amorphous hydrophobic polymer A2 is changed so as to exceed twice a number of lactic acid units (U_{A1}) contained in the hydrophobic block of the amphiphilic block polymer A1 to control the particle diameter of the molecular assembly.
(2-2) The method for controlling the particle diameter of the molecular assembly according to (2-1), wherein, a molar ratio of the amorphous hydrophobic polymer A2 to the amphiphilic block polymer A1, A2/A1 is changed.
(2-3) The method for controlling the particle diameter of the molecular assembly according to (2-1), wherein a molar ratio of the amorphous hydrophobic polymer A2 to the amphiphilic block polymer A1, A2/A1 is changed in a range of 0.1/1 to 10/1.
(2-4) The method for controlling the particle diameter of the molecular assembly according to (2-1), wherein a total number of aliphatic hydroxy acid units (TU_{A2}) contained in ail the amorphous hydrophobic polymers A2 constituting the molecular assembly is changed against a total number of lactic acid units (TU_{A1}) contained in the hydrophobic blocks of all the amphiphilic block polymers A1 constituting the molecular assembly.

Here, the number of aliphatic hydroxy acid units contained in all the amorphous hydrophobic polymers A2 constituting the molecular assembly is represented as TU_{A2}, and the number of lactic acid units contained in the hydrophobic blocks of all the amphiphilic block polymers A1 constituting the molecular assembly is represented as TU_{A1}.
(2-5) The method for controlling the particle diameter of the molecular assembly according to (2-1), wherein a total number of aliphatic hydroxy acid units (TU_{A2}) contained in ail the amorphous hydrophobic polymers A2 constituting the molecular assembly is changed so as to be equal to or larger than twice a total number of lactic acid units (TU_{A1}) contained in the hydrophobic blocks of ail the amphiphilic block polymers A1 constituting the molecular assembly [2<TU₂/TU_{A1}].

### Effects of the Invention

The molecular assembly according to the present invention comprises: an amphiphilic block polymer A1 comprising a hydrophilic block having a aaroosine unit and a hydrophobic block having a lactic acid unit; and an amorphous hydrophobic polymer A2 having an aliphatic hydroxy acid unit. In the molecular assembly, the number of aliphatic hydroxy acid units (U_{A2}) contained in the amorphous hydrophobic polymer A2 exceeds twice the number of lactic acid units (U_{A1}) contained in the hydrophobic block of the amphiphilic block polymer A1 [U_{A2}>2·U_{A1}]. This lactosome molecular assembly is considered to be formed as a micelle by self-assembly of the amphiphilic block polymer A1 and the hydrophobic polymer A2. More specifically, the hydrophilic block chain of the amphiphilic block polymer A1 forms a shell part, and the hydrophobic block chain of the amphiphilic block polymer A1 forms a core part, and the hydrophobic polymer A2 is located in the hydrophobic core due to affinity for the hydrophobic block chain. The hydrophobic polymer A2 is a hydrophobic polymer whose number of aliphatic hydroxy acid units (U_{A2}) exceeds twice the number of lactic acid units (U_{A1}) contained in the hydrophobic block of the amphiphilic block polymer A1 [U_{A2}>2·U_{A1}], and therefore has a longer chain length than the hydrophobic block of A1. The hydrophobic polymer A2 is amorphous and is therefore present in a random-coil conformation. For this reason, in spite of being a long-chain polymer, the hydrophobic polymer A2 can be stably present in the hydrophobic core. Therefore, the hydrophobic polymer A2 increases the volume of the hydrophobic core and, at the same time, increases the particle diameter of the micelle. However, neither vesicle-like particles nor rod-like particles other than micelles are excluded.

The volume of the random coil-like hydrophobic polymer A2 can be changed by changing the number of aliphatic hydroxy acid units (U_{A2}) of the hydrophobic polymer A2 under the condition that the number exceeds twice the number of lactic acid units (U_{A1}) contained in the hydrophobic block of the amphiphilic block polymer A1 [U_{A2}>2·U_{A1}]. Therefore, changing the number of aliphatic hydroxy acid units (U_{A2}) of the hydrophobic polymer A₂, that is, changing the length of the hydrophobic polymer A2 makes it possible to increase or decrease the volume of the hydrophobic core and, at the same time, to control the particle diameter of the micelle.

As described above, controlling each of the structural units of the amphiphilic block polymer A1 and the amorphous hydrophobic polymer A2 makes it possible to continuously control the particle diameter of the molecular assembly according to the present invention in a wider range of, for example, 10 to 1,000 nm and to obtain lactosome particles uniform in particle diameter.

Further, even when the same amphiphilic block polymer A1 is used, the particle diameter of the molecular assembly according to the present invention can be continuously controlled by using a different type of the amorphous hydrophobic polymer A2 together with the same amphiphilic block polymer A1, that is, by changing the number of aliphatic hydroxy acid units (U_{A2}) of the hydrophobic polymer A2. The present invention is very advantageous in that the particle diameter of the molecular assembly can be continuously controlled by changing the amorphous hydrophobic polymer A2 even when the amphiphilic block polymer A1 whose synthesis requires greater effort is not changed.

According to the present invention, it is possible to provide, in various fields such as pharmaceutical drugs, agricultural chemicals, cosmetics, food products, and electronics (e.g., battery materials), a molecular assembly having any nano-sized particle diameter depending on its intended use and application. It is also possible to provide, in the above various fields, a nano-carrier for delivering various substances using the molecular assembly having any nano-sized particle diameter depending on its intended use and application. For example, in general, a DDS carrier for cosmetics preferably has a larger particle diameter than that for medical use. According to the present invention, it is possible to provide a molecular assembly having a nano-sized particle diameter suitable for such an application. As described above, the molecular assembly according to the present invention can be used for various applications.

More particularly, according to the present invention, it is possible to provide a molecular assembly that is less likely to accumulate in tissue other than cancer tissue and is highly safe for a living body by controlling the particle diameter thereof, and it is also possible to provide a nano-carrier using the molecular assembly for use in delivering a drug or a labeling agent.

Further, according to the present invention, it is possible to more widely control the in-vivo dynamic behavior of the molecular assembly by changing the particle diameter of the molecular assembly. More specifically, when the molecular assembly having a certain particle diameter has high retentivity in certain tissue (e.g., cancer tissue), but the molecular assembly having another particle diameter has low retentivity in certain tissue (e.g., cancer tissue), the use of these molecular assemblies can expand the application of the nano-carrier using the molecular assembly for use in delivering a drug or a labeling agent, and the range of choices for its administration route. In general, a molecular assembly having a relatively large nano-sized particle diameter can have a large amount of drug encapsulated therein. Therefore, it is possible to provide a sustained-release nano-carrier for drug delivery by controlling sustained reasonability.

### BRIEF DESCRIPTION OF THE DRAWINGS

FIG. 1 shows the measurement result of DLS of No. 1 lactosome nanoparticles in Example 1.

FIG. 2 shows the measurement result of DLS of No. 2 lactosome nanoparticles in Example 1.

FIG. 3 shows the measurement result of DLS of No. 3 lactosome nanoparticles in Example 1.

FIG. 4 shows the measurement result of DLS of No. 4 lactosome nanoparticles in Example 1.

FIG. 5 shows the measurement result of DLS of No. 5 lactosome nanoparticles in Example 1.

FIG. 6 shows the measurement result of DLS of No. 6 lactosome nanoparticles in Example 1.

FIG. 7 shows the measurement result of DLS of No. 11 lactosome nanoparticles in Comparative Example 1.

FIG. 8 shows the measurement result of DLS of No. 12 lactosome nanoparticles in Comparative Example 1.

FIG. 9 shows the measurement result of DLS of No. 13 lactosome nanoparticles in Comparative Example 1.

FIG. 10 shows the measurement result of DLS of No. 14 lactosome nanoparticles in Comparative Example 1.

FIG. 11 shows the measurement result of DLS of No. 15 lactosome nanoparticles in Comparative Example 1.

FIG. 12 shows the measurement result of DLS of No. 16 lactosome nanoparticles in Comparative Example 1.

FIG. 13 is a graph showing a change in the particle diameter of lactosome nanoparticles in Example 1 and a change in the particle diameter of lactosome nanoparticles in comparative Example 1.

### MODES FOR CARRYING OUT THE INVENTION

A molecular assembly according to the present invention (Lactosome) comprises: an amphiphilic block, polymer A1 comprising a hydrophilic block having a sarcosine unit and a hydrophobic block having a lactic acid unit; and an amorphous hydrophobic polymer A2 having an aliphatic hydroxy acid unit. In the molecular assembly, the number of aliphatic hydroxy acid units (U_{A2}) contained in the amorphous hydrophobic polymer A1 exceeds twice the number of lactic acid units (U_{A1}) contained in the hydrophobic block of the amphiphilic block polymer A1 [U_{A2}>2·U_{A1}]. Description will be made below.
(1. Amphiphilic Block Polymer A₁)

The amphiphilic block polymer A1 comprises a hydrophilic block having a sarcosine unit, and a hydrophobic block having a lactic acid unit.
(1-1. Hydrophilic Block Chain)

In the present invention, the specific degree of the physical property, “hydrophilicity” of the hydrophilic block chain is not particularly limited, but, at least, the whole hydrophilic block chain shall be relatively more hydrophilic than the hydrophobic block chain having a lactic acid unit that will be described later. Alternatively, the hydrophilic block chain shall be hydrophilic to such an extent that a copolymer composed of the hydrophilic block chain and the hydrophobic block chain can have amphiphilicity as a whole molecule of the copolymer. Alternatively, the hydrophilic block chain shall be hydrophilic to such an extent that the amphiphilic block polymer can self-assemble in a solvent to form a self-assembly, especially a particulate self-assembly.

In the present invention, the hydrophilic block of the amphiphilic block polymer may have a linear structure or a branched structure. When the hydrophilic block has a branched structure, each of the branches of the hydrophilic block contains sarcosine.

The kind and ratio of a structural unit constituting the hydrophilic block are appropriately determined by those skilled in the art so that a resultant block can have such hydrophilicity as described above as a whole. For example, the hydrophilic block contains 2 to 300 sarcosine units in total. Specifically, when the hydrophilic block has a linear structure, the total number of sarcosine units may be, for example, about 10 to 300, 20 to 200, or 20 to 100. If the number of structural units exceeds the above range, when a molecular assembly is formed, the resultant molecular assembly tends to lack stability. If the number of structural units is less than the above range, a resultant block polymer cannot serve as an amphiphilic block polymer or formation of a molecular assembly tends to be difficult per se.

When the hydrophilic block has a branched structure, the total number of sarcosine: units contained in all the branches may be, for example, 2 to 200, 2 to 100, or 2 to 10. Alternatively, the total number of sarcosine units contained in all the hydrophilic blocks may be, for example, 30 to 200 or 50 to 100. The average number of sarcosine units per one branch may be, for example, 1 to 60, 1 to 30, 1 to 10, or 1 to 6. That is, each of the hydrophilic blocks can be formed to contain sarcosine or a polysarcosine chain. If the number of structural units exceeds the above range, when a molecular assembly is formed, the resultant molecular assembly tends to lack stability. If the number of structural units is less than the above range, a resultant block polymer cannot serve as an amphiphilic block polymer or formal ion of a molecular assembly tends to be difficult per se.

When the hydrophilic block has a branched structure, the number of branches of the hydrophilic block shall be 2 or more, but is preferably 3 or more from the viewpoint of efficiently obtaining a particulate micelle when a molecular assembly is formed. The upper limit of the number of branches of the hydrophilic block is not particularly limited, but is, for example, 27. Particularly, in the present invention, the number of branches of the hydrophilic block is preferably 3. The branched structure can be appropriately designed by those skilled in the art.

Sarcosine (i.e., N-methylglycine) is highly water-soluble, and a sarcosine polymer is highly flexible, because the polymer has an N-substituted amide and therefore can be more easily cis-trans isomerised as compared to a normal amide group, and steric hindrance around, the C^{α} carbon atom is low. The use of such a structure as a constituent block is very useful in that the block can have high hydrophilicity as its basic characteristic, or both high hydrophilicity and high flexibility as its basic characteristics.

Further, the hydrophilic block preferably has a hydrophilic group (typified by, for example, a hydroxyl group) at its end (i.e., at the end opposite to a linker part).

In the polysarcosine chain, all the sarcosine units may be either continuous or discontinuous. However, it is preferred that the polypeptide chain is molecularly-designed so that the basic characteristics thereof described above are not impaired as a whole.

When the hydrophilic block chain has another structural unit other than a sarcosine unit, such another structural unit is not particularly limited, but may be amino acid (including hydrophilic amino acids and other amino acid). It is to be noted that the term “amino acid” used in this specification includes natural amino acids, unnatural amino acids, and derivatives thereof obtained by modification and/or chemical alteration. Further, in this specification, the term “amino acid” includes α-, β-, and γ-amino acids. Among them, α-amino acids are preferred. Examples of α-amino acids include serine, threonine, lysine, a spar tic acid, glutamic acid, and the like.

Further, the amphiphilic block polymer A1 may further have a group such as a sugar chain or polyether. In this case, the amphiphilic block polymer A1 is preferably molecularly-designed so that the hydrophilic block has a sugar chain or polyether.
(1-2. Hydrophobic Block)

In the present invention, the specific degree of the physical property, “hydrophobicity” of the hydrophobic block is not particularly limited, but, at least, the hydrophobic block shall be hydrophobic enough to be a region relatively more hydrophobic than the whole hydrophilic block so that a copolymer composed of the hydrophilic block and the hydrophobic block can have amphiphilicity as a whole molecule of the copolymer, or so that the amphiphilic block polymer can self-assemble in a solvent to form, a self-assembly, preferably a particulate self-assembly.

The hydrophobic block present in one amphophilic block polymer may or may not be branched. However, it is considered that when the hydrophobic block is not branched, a stable core/shell-type molecular assembly having a smaller particle diameter can be easily formed, because a hydrophilic branched shell part is denser than a hydrophobic core.

In the present invention, the hydrophobic block contains a lactic acid unit. The kind and ratio of a structural unit constituting the hydrophobic block are appropriately determined by those skilled in the art so that a resultant block can have such hydrophobicity as described above as a whole. For example, the number of lactic acid units (U_{A1}) contained in the hydrophobic block is 5 to 400. Specifically, for example, when the hydrophobic block is not branched, the number of lactic acid units may be, for example, 5 to 100, 15 to 60, or 25 to 45. When the hydrophobic block is branched, the total number of lactic acid units contained in all the branches may be, for example, 10 to 400, preferably 20 to 200. In this case, the average number of lactic acid units per one branch is, for example, 5 to 100, preferably 10 to 100.

If the number of structural units exceeds the above range, when a molecular assembly is formed, the resultant molecular assembly tends to lack stability. If the number of structural units is less than the above range, formation of a molecular assembly tends to be difficult per se.

When the hydrophobic block is branched, the number of branches is not particularly limited, but may be, for example, equal to or less than the number of branches of the hydrophilic block from the viewpoint of efficiently obtaining a particulate micelle when a molecular assembly is formed.

Polylactic acid has the following basic characteristics.

Polylactic acid has excellent biocompatibility and stability. Therefore, a molecular assembly obtained from an amphiphilic material having such polylactic acid as a constituent block is very useful from the viewpoint of applicability to a living body, especially a human body.

Further, polylactic acid is rapidly metabolized due to its excellent biodegradability, and is therefore less likely to accumulate in tissue other than cancer tissue in a living body. Therefore, a molecular assembly obtained from an amphiphilic material having such polylactic acid as a constituent block is very useful from the viewpoint of specific accumulation in cancer tissue.

Further, polylactic acid is excellent in solubility in low-boiling point solvents. This makes it possible to avoid the use of a hazardous high-boiling point solvent when a molecular assembly is obtained from an amphiphilic material having such polylactic acid as a constituent block. Therefore, such a molecular assembly is very useful from the viewpoint of safety for a living body.

It is to be noted that, in a polylactic acid chain (FLA) constituting the hydrophobic block, all the lactic acid units may be either continuous or dascontinuous. However, it is preferred that the hydrophobic block is moleoularly-designed so that the basic characteristics described above are not impaired as a whole.

The polylactic acid chain (PLA) constituting the hydrophobic block may be either a poly L-lactic acid chain (PLLA) constituted from L-lactic acid units, or a poly D-lactic acid chain (PDLA) constituted from D-lactic acid units. Alternatively, the PLA may be constituted from both L-lactic acid units and D-lactic acid units. In this case, the arrangement of L-lactic acid units and D-lactic acid units may be any one of alternate arrangement, block arrangement, and random arrangement.

When the hydrophobic block chain has another structural unit other than a lactic acid unit, the kind and ratio of such another structural unit are not particularly limited as long as a resultant block chain can have such hydrophobicity as described above as a whole, but the hydrophobic block chain is preferably molecularly-designed to have desired various characteristics.

When, the hydrophobic block chain has another structural unit other than a lactic acid unit, such another structural unit can be selected from, the group consisting of hydroxy acids other than lactic acid and amino acids (including hydrophobic amino acids and other amino acids). Examples of hydroxy acids include, but are not limited to, glycolic acid, hydxoxyisobutyric acid, and the like. Many of hydrophobic amino acids have an aliphatic side chain, an aromatic side chain, and the like. Examples of natural amino acids include glycine, alanine, valine, leucine, isoleucine, proline, methionine, tyrosine, tryptophan, and the like. Examples of unnatural amino acids include, but are not limited to, amino acid derivatives such as methyl glutamate, benzyl glutamate, methyl aspartate, ethyl aspartate, and benzyl aspartate.
(1-3. Synthesis of Amphiphilic Block Poxymer A1)

In the present invention, a method for synthesizing the amphiphilic block polymer A1 is not particularly limited, and a known, peptide synthesis method, a known polyester synthesis method, and/or a known depsipeptide synthesis method can be used.

Peptide synthesis can be performed by, for example, ring-opening polymerization of N-carboxyamino acid anhydride (amino acid NCA) using, as an initiator, a base such as an amine.

Polyester synthesis can be performed by, for example, ring-opening polymerization of lactide using, as an initiator, a base such as an amine or a metal complex. The type of lactide can be appropriately determined by those skilled in the art in consideration of the desired optical purity of a resultant block chain. For example, the type of lactide can be appropriately selected from L-lactide, D-lactide, DL-lactide and mesolactide, and the amount of lactide to be used can be appropriately determined by those skilled in the art depending on the desired optical purity of a resultant block chain.

Depsipeptide synthesis can be performed by, for example, a method in which polylactic acid is first synthesized as a hydrophobic block and then a polypeptide chain is extended as a hydrophilic block; or a method in which a polypeptide chain is first synthesized as a hydrophilic block and then polylactic acid is extended as a hydrophobic block.

In the molecular assembly according to the present invention, the chain length of polylactic acid can be adjusted. From the viewpoint of more flexibly controlling the chain length of polylactic acid, synthesis of the amphiphilic block polymer A1 is preferably performed by a method in which polylactic acid is first synthesized as a hydrophobic block and then a polypeptide chain is extended as a hydrophilic block chain. Further, the polymerization degree of polylactic acid as a hydrophobic block chain in the amphiphilic block polymer A1 can be more easily and accurately controlled than that of polysarcosine as a hydrophilic block chain.

WO 2009/148121 A (linear type) and WO 2012/176885 A (branched type) can be referred to for the structure and synthesis of the amphiphilic block polymer A1.
(2. Amorphous Hydrophobic Polymer A2)

The hydrophobic polymer A2 is a hydrophobic aliphatic polymer having an aliphatic hydroxy acid unit and is an amorphous polymer. In this specification, the amorphous polymer refers to a polymer whose melting point is not measured in accordance with JIS K7121. The specific degree of physical property, “hydrophobia city” of the hydrophobic polymer A2 is not particularly limited, but at least, the hydrophobic polymer A2 shall be relatively more hydrophobic than the hydrophilic block of the amphiphilic block polymer A1.

Examples of aliphatic hydroxy acid constituting the hydrophobic polymer A2 include, but are rot limited to, lactic acid, glycolic acid, hydroxyisobutyric acid, and the like.

From the viewpoint of miscibility with the hydrophobic block of the amphiphilic block polymer A1, the hydrophobic polymer A2 preferably has, as an aliphatic hydroxy acid unit, at least one selected from the group consisting of a lactic acid unit and a glycolic acid unit. More specifically, the hydrophobic polymer A2 is preferably a lactic acid homopolymer, or a copolymer of lactic acid and glycolic acid (alternate arrangement, block arrangement, or random, arrangement).

A polylactic acid chain (PLA) constituting the hydrophobic polymer A2 is constituted from both L-lactic acid units and D-lactic acid units so as to be amorphous. In this case, the arrangement of L-lactic acid units and D-lactic acid units may be any one of alternate arrangement, block arrangement, and random arrangement.

When the hydrophobic polymer A2 is a copolymer of lactic acid and another aliphatic hydroxy acid (e.g., glycolic acid), lactic acid units may include only L-lactic acid units or only D-lactic acid units. Further, when the hydrophobic polymer A2 is a copolymer of lactic acid, and glycolic acid, the ratio between a lactic acid unit and a glycolic acid unit may be taken into consideration, because when the ratio of a glycolic acid unit increases, the solubility of a resultant copolymer in an organic solvent tends to decrease.

The number of aliphatic hydroxy acid units (U_{A2}) contained in the hydrophobic polymer A2 exceeds twice the number of lactic acid units (U_{A1}) contained in the hydrophobic block of the amphiphilic block polymer A1 [U_{A2}>2·U_{A1}]. Under this condition, the hydrophobic polymer A2 preferably has 35 or more aliphatic hydroxy acid units, and more preferably has 60 or more aliphatic hydroxy acid units. The hydrophobic polymer A2 sometimes has 200 or more aliphatic hydroxy acid units.

The hydrophobic polymer A2 can be synthesized by a polymerization method known to those skilled in the art. For example, amorphous polylactic acid may be synthesized by ring-opening polymerization of lactide with reference to WO 2009/148121 A ([0239], [0241]). Alternatively, amorphous polylactic acid may be synthesized by direct polymerization of lactic acid. Further, an amorphous copolymer of lactic acid and glycolic acid may be synthesized by ring-opening polymerization of lactide and glycolide. Alternatively, an amorphous copolymer of lactic acid and glycolic acid may be synthesized by direct polymerization of lactic acid and glycolic acid.
(3. A1/A2 Molecular Assembly)

In the molecular assembly (Lactosome), the number of aliphatic hydroxy acid units (U_{A2}) contained in the amorphous hydrophobic polymer A2 exceeds twice the number of lactic acid units (U_{A1}) contained in the hydrophobic block of the amphiphilic block polymer A1 (U_{A2}>2·U_{A1}). That is, the hydrophobic polymer A₂ is a hydrophobic polymer whose number of aliphatic hydroxy acid units (U_{A2}) exceeds twice the number of lactic acid units (U_{A1}) contained in the hydrophobic block of the amphiphilic block polymer A1, and therefore has a longer hydrophobic chain length than the hydrophobic block of A1. The hydrophobic polymer A2 is amorphous and is therefore present in a random-coil conformation. For this reason, in spite of being a long-chain polymer, the hydrophobic polymer A2 can be stably present in the hydrophobic core. Therefore, the hydrophobic polymer A2 increases the volume of the hydrophobic core and, at the same time, increases the particle diameter of the molecular assembly. If the number of aliphatic hydroxy acid units in A2 is equal to or less than twice the number of lactic acid units contained in the hydrophobic block of the amphiphilic block polymer A1, a resultant polymer A2 is poor in the effect of increasing the volume of core of a micelle formed by the amphiphilic block polymer A1. Therefore, the resultant polymer A2 has a poor ability to control the particle diameter of a micelle originally formed by the amphiphilic block polymer A1.

This lactosome molecular assembly is considered to as formed as a micelle by self-assembly from the amphiphilic block polymer A1 and the hydrophobic polymer A2. More specifically, the hydrophilic block chain of the amphiphilic block polymer A1 forms a shell part, and the hydrophobic block chain of the amphiphilic block chain A1 forms a core part, and the hydrophobic polymer A2 is located in the hydrophobic core due to affinity for the hydrophobic block chain. It is considered that the volume of hydrophobic core of a micelle originally formed by the amphiphilic block polymer A1 (in the absence of the hydrophobic polymer A2) depends on the chain length of the hydrophobic block chain of A1, that is, on the number of lactic acid units (U_{A1}) contained in the hydrophobic block of A1. That is, it is considered that when the chain length of the hydrophobic block of A1 is longer, the volume of the hydrophobic core is larger, and when the chain length of the hydrophobic block of A1 is shorter, the volume of the hydrophobic core is smaller. Therefore, it is considered that in order to increase the volume of the hydrophobic core, it is necessary to allow the long-chain hydrophobic polymer A2 to be present depending on the chain length of the hydrophobic block. For this reason, in the lactosome molecular assembly according to the present invention, the number of aliphatic hydroxy acid units (U_{A2}) contained in the amorphous hydrophobic polymer A2 exceeds twice the number of lactic acid units (U_{A1}) contained in the hydrophobic block of the amphiphilic block polymer A1 (U_{A2}>2·U_{A1}).

For example, the particle diameter of the lactosome molecular assembly may be controlled by changing the number of aliphatic hydroxy acid units (U_{A2}) contained in the amorphous hydrophobic polymer A2 so that U_{A2} exceeds twice the number of lactic acid units (U_{A1}) contained in the hydrophobic block of the amphiphilic block polymer A1 but is equal to or less than ten times U_{A1}.

The volume of the random coil-like hydrophobic polymer can be changed by changing the number of aliphatic hydroxy acid units (U_{A2}) of the hydrophobic polymer A2 under the condition that U_{A2} exceeds twice the number of lactic acid units (U_{A1}) contained in the hydrophobic block of the amphiphilic block polymer A1. Therefore, changing the number of aliphatic hydroxy acid units (U₂) of the hydrophobic polymer A2, that is, changing the length of the hydrophobic polymer A2 makes it possible to increase or decrease the volume of the hydrophobic core and, at the same time, to control the particle diameter of the micelle. It is to foe noted that the volume of hydrophobic core of a micelle originally formed by the amphiphilic block polymer A1 (in the absence of the hydrophobic polymer A₂) is a minimum volume, which gives a minimum, particle diameter of the micelle.

The particle diameter of the lactosome molecular assembly may be controlled also by changing the molar ratio A2/A1 of the amorphous hydrophobic polymer A2 to the amphiphilic block polymer A1.

For example, the particle diameter of the lactosome molecular assembly may be controlled by changing the molar ratio A2/A1 of the amorphous hydrophobic polymer A2 to the amphiphilic block polymer A1, in the range of 0.1/1 to 10/1.

The particle diameter of the lactosome molecular assembly may be controlled also by changing the total number of aliphatic hydroxy acid units (TU_{A2}) contained in all the amorphous hydrophobic polymers A2 constituting the molecular assembly against the total number of lactic acid units (TU_{A1}) contained in the hydrophobic blocks of all the amphiphilic block polymers A1 constituting the molecular assembly.

For example, the particle diameter of the lactosome molecular assembly may be controlled by changing the total number of aliphatic hydroxy acid units (TU_{A2}) contained in all the amorphous hydrophobic polymers A2 constituting the molecular assembly so that TU_{A2} is equal to or larger than twice the total number of lactic acid units (TU_{A1}) contained in the hydrophobic blocks of all the amphiphilic block polymers A1 constituting the molecular assembly but is equal to or less than ten times TU_{A1}.

As described above, controlling each of the structural units of the amphiphilic block polymer A1 and the amorphous hydrophobic polymer A2 makes it possible to continuously control the particle diameter of the molecular assembly according to the present invention in a wider range of, for example, 10 to 1,000 nm and to obtain lactosome particles uniform in particle diameter. As described above, WO 2009/148121 A discloses that the particle diameter of lactosome is 10 nm to 500 nm in paragraph [0127], but the particle diameter of the lactosome actually demonstrated is only 30 nm to 130 nm disclosed in paragraph [0251], and continuous control of the particle diameter in a wide range is not disclosed.

A method for measuring the size of the molecular assembly according to the present invention is not particularly limited, and is appropriately selected by those skilled in the art. Examples of such a method include an observational method with a TEM (Transmission Electron Microscope) and a DLS (Dynamic Light Scattering) method. In the case of a DLS method, the translational diffusion coefficlent of particles undergoing Brownian movement in a solution is measured.
(4. Embodiment having Functional Structure)

The molecular assembly according to the present invention can have a functional structure that allows the molecular assembly to have a useful form or function for use in a molecular imaging system or a drug delivery system. Therefore, the molecular assembly according to the present invention can be a structure useful as a probe for molecular imaging or a preparation for a drug delivery system. The same goes for other applications such as cosmetics.

Examples of a specific embodiment of the molecular assembly having a functional structure Include an embodiment in which a functional group selected from the group consisting of a signal group and a ligand group is bound to the amphiphilic block polymer itself constituting the molecular assembly, and an embodiment in which the molecular assembly encapsulates a functional substance selected from the group consisting of a signal agent and a drug.
(4-1. Binding of functional Group)

The functional group Is, for example, an organic group, and is appropriately selected by those skilled in the art depending on the intended use of the molecular assembly. Examples of the functional group include a signal group and a ligand group.

A signal group is a group having a property detectable for imaging. Examples of such a signal group include fluorescent groups, radioactive element-containing groups, and magnetic groups, Means for detecting these groups may be appropriately selected by those skilled, in the art.

Examples of the fluorescent groups include, but are not limited to, groups derived from fluorescein-based dyes, cyanine-based dyes such as indocyanine dyes, rhodamine-based dyes, and quantum dots. In the present invention, near-infrared fluorescent groups (e.g., groups derived from cyanine-based dyes or quantum dots) may be used.

Each substituent group having a hydrogen bond exhibits absorption in the near-infrared region (700 to 1,300 nm), but the degree of absorption is relatively small. Therefore, near-infrared light easily penetrates through living tissue. It can be said that by utilizing such characteristics of near-infrared light, in-vivo information can be obtained without putting an unnecessary load on the body. Particularly, when a target to be measured is decided to a small animal, or a site close to the body surface of an animal, near-infrared fluorescence can give useful information.

More specific examples of the near-infrared fluorescent groups include groups derived from indocyanine dyes such as ICG (indocyanine green) , Cy7, DY776, DY750, Alexa790, Alexa750, and the like. In a case where the molecular assembly according to the present invention is intended for use targeting, for example, cancer, groups derived from an indocyanine dye such as ICG may be particularly preferably used from the viewpoint of accumulation in a cancer.

Examples of the radioactive element-containing groups include, but are not limited to, groups derived from saccharides, amino acids, or nucleic acids labeled with a radioisotope such as ¹⁸F. One specific example of a method for introducing a radioactive element-containing group includes a method comprising the step of polymerizing lactide using mono-Fmoc (9-fluorenylmethyloxycarbonyl) ethylenediamine, the step of protecting a terminal OH group by a silyl protecting group, the step of eliminating Fmoc by piperidine treatment, the step of polymerizing sarcosine-N-carboxyanhydride (SarNCA) and terminating the end of the polymer, the step of eliminating the silyl protecting group to perform conversion to a sulfonate ester (e.g., trifluoromathanesulfonate ester, p-toluenesuifonate ester), and the step of introducing a radioactive element-containing group. If necessary, this specific example may be modified by those skilled in the art.

Examples of the magnetic groups include, but are not limited to, groups having a magnetic substance such as ferrichrome and groups contained in ferrite nanoparticles and magnetic nanoparticles.

The ligand group shall be one that binds to a biomolecule expressed in a target cell to control the directivity of the molecular assembly to thereby improve the targeting property of the molecular assembly. Examples of the ligand group include an antibody, a cell-adhesive peptide, a sugar chain, a water-soluble polymer, and the like.

Examples of the antibody include those having an ability to specifically bind to an antigen expressed in a cell in a target site.

Examples of the cell-adhesive peptide include adhesion factors such as RGD (arginine-glycine-aspartic acid).

Examples of the sugar chain include stabilizers such as carboxymethyl cellulose and amylase, and those having an ability to specifically bind to a protein expressed in a cell in a target site.

Examples of the water-soluble polymer include polymers such as polyether chains and polyvinyl alcohol, chains.

Such a group can be preferably bound to the terminal structural unit of the hydrophilic block-side in the amphiphilic block polymer. This makes it possible, when a micelle is formed, to obtain a particle having the functional groups on its surface, that is, a particle having a surface modified with the functional groups.
(4-2. Encapsulation of Functional Substance)

The functional substance is selected from the group consisting of a signal agent and a drug. This substance is a hydrophobic compound and is encapsulated by placing said substance in the hydrophobic core of the molecular assembly.

As the signal agent, a molecule having the above-described signal group can be used. Among such molecules, near-infrared fluorescent substances such as indocyanine green-based dyes, or radioactive element-containing substances such as saccharides, amino acids, or nucleic acids labeled with a radioisotope such as ¹⁸F may be preferably used in the present invention.

As the drug, one suitable for a target disease is appropriately selected by those skilled in the art. Specific examples of the drug include anticancer drugs, antimicrobial agents, antiviral agents, anti-inflammatory agents, immunosuppressive drugs, steroid drugs, hormone drugs, anti-angiogenic agents, and the like. These drug molecules may be used singly or in combination, of two or more thereof.

The functional substance to foe encapsulated may be bound with a polyaliphatic hydroxy acid group. The polyaliphatic hydroxy acid group is not particularly limited, but is a group comprising, for example, a lactic acid unit, a glycolic acid unit, or a hydroxyisobutyric acid unit as a main component, and is preferably a group comprising a lactic acid unit and/or a glycolic acid unit as a main component(s). All the aliphatic hydroxy acid units such as lactic acid units may be either continuous or discontinuous. The structure, chain, length, and optical purity of the aliphatic hydroxy acid group can be basically determined from the same viewpoint as described above with reference to the molecular design of the hydrophobic block. This also makes it possible to obtain the effect that the functional substance can have excellent affinity for the hydrophobic block of the amphiphilic block polymer in the molecular assembly.

The amount of the functional substance encapsulated is not particularly limited, but when the functional substance is, for example, a fluorescent substance, the amount of a fluorescent dye may be 0.5 to 50 mol % with respect to the total amount of the amphiphilic block polymer and the fluorescent dye. The same can be applied to the amount of another functional substance (a radioactive substance as an example) encapsulated,
(5. Formation of Molecular Assembly)

A method for forming the molecular assembly (lactosome) is not particularly limited, and can be appropriately selected by those skilled in the art depending on, for example, the desired size and characteristics of the molecular assembly and the kind, properties, and amount of a functional structure to be carried by the molecular assembly. If necessary, after being formed by a method that will be described later, the resultant molecular assembly may be surface-modified by a known method. It is to be noted that whether particles have been formed or not may be confirmed toy observation with an electron microscope.
(5-1. Film Method)

The amphiphilic block polymer A1 and the amorphous hydrophobic polymer A2 used in the present invention are soluble in low-boiling point solvents, and therefore the molecular assembly can be prepared by a film method.

The film method includes the following steps of: preparing a solution, in a container (e.g., a glass container), containing the amphiphilic block polymer A1 and the amorphous hydrophobic polymer A2, and if necessary, the functional substance in an organic solvent; removing the organic solvent from the solution to obtain a film, comprising the amphiphilic block polymer A1 and the amorphous hydrophobic polymer A2, and if necessary, the functional substance on an inner wall of the container; and adding water or an aqueous solution into the container, and if necessary, performing ultrasonic treatment to convert the film into a particulate molecular assembly (having the functional substance encapsulated therein, if necessary), thereby obtaining a dispersion liquid of the molecular assembly. Further, the film, method, may include the step of subjecting the dispersion liquid of the molecular assembly to freeze-drying treatment.

The solution containing the amphiphilic block polymer A1 and the hydrophobic polymer A2, and if necessary, the functional substance in an organic solvent is appropriately prepared by those skilled in the art. For example, the solution may be prepared by mixing, at a time, all the polymers A1 and A2 that should be osed, and/or if necessary, the functional substance; or may be prepared by previously preparing a film of one or two component(s) of the polymers A1 and A2 that should be used, and/or if necessary, the functional substance (e.g., the polymer A1 component), and then adding a solution containing the other component(s) that should be used (e.g., the polymer A2, and/or if necessary, the functional substance). The previously-prepared film of one of the polymers may be formed in accordance with a method that will be described later (i.e., a method for forming a film comprising the polymer A1 and the polymer A2, and/or if necessary, the functional substance).

The organic solvent used in the film method is preferably a low-boiling point solvent. In the present invention, the term “low-boiling point solvent” refers to one whose boiling point is 100° C. or less, preferably 90° C. or less at 1 atmospheric pressure. Specific examples of such a low-boiling point solvent include chloroform, diethyl ether, acetonitrile, 2-propanol, ethanol, acetone, dichloromethane, tetrahydrofuran, hexane, and the like.

When such a low-boiling point solvent is used for dissolving the polymer A1 and the polymer A2, and/or if necessary, the functional substance, solvent removal can be very easily performed. A method for solvent removal is not particularly limited, and may be appropriately determined be those skilled in the art depending on, for example, the boiling point of an organic solvent to be used. For example, solvent removal may be performed under reduced pressure or by natural drying.

After the organic solvent is removed, a film comprising the amphiphilic block, polymer A1 and the hydrophobic polymer A2, and/or if necessary, the functional substance is formed on the inner wall of the container. Water or an aqueous solution is added to the container to which the film is attached. The water or aqueous solution is not particularly limited, and a biochemically or pharmaceutically acceptable one may be appropriately selected by those skilled in the art. Examples thereof include distilled water for injection, normal saline, a buffer solution, and the like.

After water or an aqueous solution is added, warming treatment is performed under conditions of 20 to 90° C. and 1 to 60 minutes so that the molecular assembly is formed in the process of peeling-off of the film from the inner wall of the container. After the completion of the treatment, a dispersion liquid in which the molecular assembly (when the functional substance is used, the molecular assembly having the functional substance encapsulated therein) is dispersed in the water or aqueous solution is prepared in the container. At this time, ultrasonic treatment may be performed, if necessary.

This dispersion liquid can be directly administered to a living body. That is, the molecular assembly does not need to be stored by itself under solvent-free conditions. Therefore, the dispersion liquid is very effectively applied to, for example, a PET (Positron Emission Tomography) molecular probe using a drug having a short half-life.

When the obtained dispersion liquid is subjected to freeze-drying treatment, a method for freeze-drying treatment is not particularly limited, and any known method can be used. For example, the dispersion liquid of the molecular assembly obtained in such a manner as described above may be frozen by, for example, liquid nitrogen and sublimated under reduced pressure. In this way, a freeze-dried product of the molecular assembly is obtained. That is, the molecular assembly can be stored as a freeze-dried product. If necessary, water or an aqueous solution may be added to the freeze-dried product to obtain a dispersion liquid of the molecular assembly so that the molecular assembly can be used. The water or aqueous solution is not particularly limited, and a biochemically or pharmaceutically acceptable one may be appropriately selected by those skilled in the art. Examples thereof include distilled water for injection, normal saline, a buffer solution, and the like.

Here, before subjected to freeze-drying treatment, the dispersion liquid may contain, in addition to the molecular assembly according to the present invention formed from the amphiphilic block polymer A1 and the hydrophobic polymer A2, and/or if necessary, the functional substance, the amphiphilic block polymer A1 and the hydrophobic polymer A2, and/or if necessary, the functional substance remaining as they are without forming the molecular assembly. When such a dispersion liquid is subjected to freeze-drying treatment, the molecular assembly can further be formed from the amphiphilic block polymer A1 and the hydrophobic polymer A2, and/or if necessary, the functional substance remaining without forming the molecular assembly according to the present invention in the process of concentration of the solvent. Accordingly, this makes it possible to efficiently prepare the molecular assembly according to the present invention.
(5-2. Injection Method)

An injection method includes the following steps of: preparing a solution, in a container (e.g., a test tube), containing the amphiphilic block polymer A1 and the amorphous hydrophobic polymer A2, and if necessary, the functional substance in an organic solvent; dispersing the solution in water or an aqueous solution; and removing the organic solvent. In the injection method, the step of purification treatment may be appropriately performed before the step of removing the organic solvent.

Examples of the organic solvent used in the injection method include trifluoroethanol, ethanol, 2-propanol, hexafluoroisopropanol, dimethylsulfoxide, dimethylformamide, and the like.

Examples of the water or aqueous solution used include distilled water for injection, normal saline, a buffer solution, and the like.

Examples of the purification treatment performed include gel filtration chromatography, filtering, ultracentrifligation, and the like.

When the molecular assembly to be administered to a living body is obtained in such a manner as described above using an organic solvent hazardous to a living body, removal of the organic solvent needs to be strictly performed.

When the molecular assembly is prepared as an encapsulated-type vesicle, the molecular assembly may be prepared toy dissolving or suspending a substance to be encapsulated in a water-based solvent such as distilled water for injection, normal saline, or a buffer solution to obtain an aqueous solution or suspension; and dispersing, into the aqueous solution or suspension, a solution obtained by dissolving the amphiphilic block polymer A1 and the hydrophobic polymer A2, and/or if necessary, the functional substance in the above-mentioned organic solvent.
(6. Molecular Probe)

The molecular assembly according to the present invention appropriately holding a desired molecule is useful in a molecular imaging system and a drug delivery system. In this specification, the molecular assembly intended to be used in such systems is sometimes referred to as “molecular probe” or “nanoparticle”.
(6-1. Molecular Probe for Molecular Imaging)

When the molecular assembly according to the present invention has a labeling group and/or a labeling agent, such a molecular assembly is useful as a molecular probe for molecular imaging.

Examples of the labeling group include those mentioned above. These labeling groups may toe used singly or in combination of two or more of them.

Examples of the labeling agent include a molecule having the signal group described above and a molecule having the ligand group described above. These molecules may be used singly or in combination of two or more of them.

For example, the molecular probe for molecular imaging may be of a type having a labeling agent introduced there into via a covalent bond, or of a type having a signal agent coordinated by a ligand.

In other cases, the molecular probe for molecular imaging may be of a micelle type containing a labeling agent therein, or of a vesicle type having a labeling agent-containing aqueous phase therein.

The molecular probe for molecular imaging allows the above marker to specifically accumulate in a lesion site or a diseased site, which makes if possible to perform imaging of the site.

Specific examples of the molecular probe for molecular imaging include a molecular probe for fluorescent imaging, a molecular probe for positron emission tomography (PET), a molecular probe for nuclear magnetic resonance imaging (MRI), and the like.
(6-2. Molecular Probe for Drug Delivery System)

When the molecular assembly according to the present invention has a ligand coordinating to a drug as a labeling group and/or a drug, such a molecular assembly is useful as a molecular probe for drug delivery system.

The drug to be used is not particularly limited as long as the drug is suitable for a target disease. Specific examples of the drug include anticancer drugs, antimicrobial agents, antiviral agents, anti-inflammatory agents, immunosuppressive drugs, steroid drugs, hormone drugs, anti-angiogenic agents, and the like. These drug molecules may be used singly or in combination of two or more of them.

Specific examples of the anticancer drugs include camptothecin, exatecan (camptothecin derivative), gemcitabine, doxorubicin, irinotecan, SH-38 (irinotecan active metabolite), 5-FU, cisolatin, oxaliplatin, paclitaxel, docetaxel, and the like.

For example, the molecular probe for drug delivery system may be of a type having a ligand coordinating to a drug as a labeling group introduced via a covalect bond.

In other cases, the molecular probe for drug delivery system may be of a micelle type containing a drug therein, or of a vesicle type having a drug-containing aqueous phase therein.

The molecular probe for drug delivery system allows a drug to specifically accumulate in a lesion site or a diseased site, which makes it possible to allow the drug to act on cells in the site.

The molecular assembly according to the present invention may have both a drug and a signal agent (or a signal group). In this case, the nanoparticle Is useful as a molecular probe for both a drug delivery system and a molecular imaging system.

In the preparation of the molecular assembly according to the present invention, the volume of the random coil-like hydrophobic polymer A2 can be changed by changing the number of aliphatic hydroxy acid units (U_{A2}) of the hydrophobic polymer A2 under the condition that U_{A2} exceeds twice the number of lactic acid units (U_{A1}) contained in the hydrophobic block of the amphiphilic block polymer A1 (U_{A2}>2·U_{A1}). Therefore, changing the number of aliphatic hydroxy acid units (USud of the hydrophobic polymer A2, that is, changing the length of the hydrophobic polymer A2 makes it possible to increase or decrease the volume of the hydrophobic core, and at the same time, to control the particle diameter of the micelle.

Further, the particle diameter of the lactosome molecular assembly may be control led also by changing the molar ratio A2/A1 of the amorphous hydrophobic polymer A2 to the amphiphilic block polymer A1, in the range of, for example, 0.1/1 to 10/1.

Further, the particle diameter of the lactosome molecular assembly may be controlled also by changing the total number of aliphatic hydroxy acid units (TU_{A2}) contained in all the amorphous hydrophobic polymers A2 constituting the molecular assembly so that TU_{A2} is, for example, equal to or larger than twice the total number of lactic acid units (TU_{A2}) contained in the hydrophobic blocks of all the amphiphilic block polymers A1 constituting the molecular assembly but equal to or less than ten times TU_{A2}.

By doing so, it is possible to adjust, for example, the particle diameter, shape, tissue selectivity, in-vivo degradation rate, and sustained-reasonability of an encapsulated drug or signal agent of the molecular assembly.
(7. Molecular Imaging System and Drug Delivery System)

A molecular imaging systems and a drug delivery system according to the present invention include administration of the above-described molecular assembly to a living body. These systems according to the present invention are characterized by using the above-described molecular probe, and other specific procedures can be appropriately determined by those skilled in the art based on known molecular imaging system and drug delivery system.
(7-1. Administration of Molecular Probe)

A method for administration to a living body is not particularity limited, and can be appropriately determined by those skilled in the art depending on, for example, the administration target and intended use of the molecular probe. Therefore, the administration method may be either systemic or local. That is, the molecular probe can be administered by any one of injection (needle injection or needleless injection), oral administration, and external administration.
(7-2. Administration Target)

The administration target in the molecular imaging system and drug delivery system according to the present invention is not particularly limited. Particularly, the molecular assembly according to the present invention is excellent in specific accumulation in a cancer site. The molecular assembly according to the present invention accumulates in cancer tissue due to EPR (enhanced permeability and retention) effect, and therefore its accumulation does not depend on the kind of cancer. Accordingly, the administration target of the molecular assembly according to the present invention is preferably a cancer. Examples of the cancer as the administration target include a wide variety of cancers such as a liver cancer, a pancreas cancer, a lung cancer, a uterine cervical cancer, a breast cancer, and a colon cancer.

Further, the specific accumulation of the molecular assembly according to the present invention in a cancer site is particularly due largely to the realisation of rapid metabolism in the liver. Therefore, the molecular assembly according to the present invention is very effective when its administration target is a liver cancer or a cancer that may occur around the liver.
(7-3. Detection of Molecular Probe)

The molecular imaging system according to the present invention further includes the step of detecting the administered molecular probe. By detecting the administered molecular probe, it is possible to observe the appearances of the administration target (especially, the position and size of, for example, cancer tissue) from outside the body.

As a detection method, any means that can visualise the administered molecular assembly can be used. The means can be appropriately determined by those skilled in the art depending on the kind of signal group or signal agent of the molecular probe.

For example, in the case of fluorescent imaging, a living body to which the molecular probe has been administered is irradiated with excitation light to detect a signal such as fluorescence derived from the signal, group or signal agent of the molecular probe in the body.

Parameters such as excitation wavelength and fluorescence wavelength to be detected can be appropriately determined by those skilled in the art depending on the kind of signal group or signal agent of the molecular probe to be administered and the kind of administration target.

In the of positron emission tomography (PET), annihilation γ-rays emitted from the signal, group or signal agent of the molecular probe in the body can be detected by a γ-ray detector.

in the case of nuclear magnetic resonance imaging (MRI), a local magnetic field distortion produced by a magnetic material of the signal group or signal agent of the molecular probe in the body can be detected as a change in MRI signal by a receiver coil.

The time from administration to the start of detection can be appropriately determined by those skilled in the art depending on the kind of signal group or signal agent of the molecular probe to be administered, and the kind of administration target. For example, in the case of fluorescent imaging, detection may be started after 3 to 48 hours from administration, and in the case of FET or MRI, detection may be started after 1 to 9 hours from administration. If the time is shorter than the above range, a detected signal is too strong, and therefore it tends to be difficult to clearly distinguish an administration target from other sites (background). On the other hand, if the time is longer than the above range, the molecular probe tends to be excreted from an administration target.

From the viewpoint of accuracy, detection of the molecular probe is preferably performed by measuring a living body not from one direction but from two or more directions. Specifically, a living body may be measured from at least three directions, more preferably from at least five directions. When measurement is performed from five directions, a living body can be measured from, for example, both right and left abdomen sides, both right and left sides of the body, and a back side.
(7-4. Stability of Lactosome in Blood)

The molecular probe according to the present invention is excellent in stability in blood.

More specifically, the molecular probe according to the present invention has blood retentivity at least comparable to that of a nanoparticle modified by a water-soluble polymer compound, polyethylene glycol (PEG) conventionally known as a nanoparticle having excellent properties. A method for measuring the lactosome in blood can be appropriately determined by those skilled in the art depending on the kind of signal group or signal agent of the molecular probe.

### EXAMPLES

Hereinbelow, the present invention will be described in more detail with reference to examples, but the present invention is not limited thereto.

### (Amphiphilic Block Polymer A1)

The amphiphilic block polymer A1 can be synthesized with reference to a method described in WO 2009/148121 A and WO 2012/176885 A.

### (PSar63-PLLA30)

As shown by the following chemical formula, aminated poly-L-lactic acid (a-PLLA) (average polymerization degree: 30) was first synthesized using L-lactide (compound 1) and N-carbobenzoxy-1,2-diaminoethane hydrochloride (compound 2).

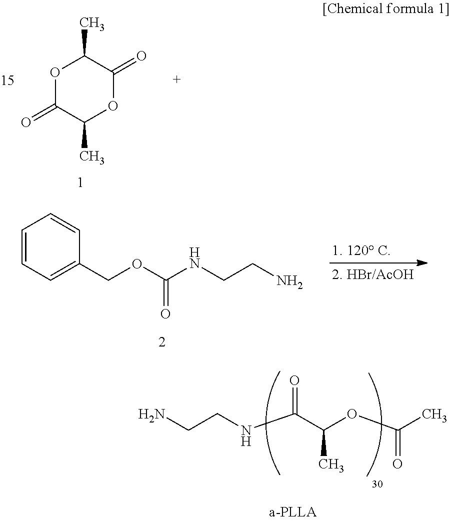

Then, sarcosine-NCA (Sar-NCA) and aminated poly-L-lactic acid (a-PLLA) were reacted using glycolic acid, O-(benzotriazol-1-yl)-N,N,N′, N′-tetramethyluronium hexafluorophosphate (HATU), and N,N-diisopropylethylamine (DIEA) to synthesize a linear amphiphilic block polymer (PSar63-PLLA30) comprising a hydrophilic block having 63 sarcosine units and a hydrophobic block having 30 L-lactic acid units.

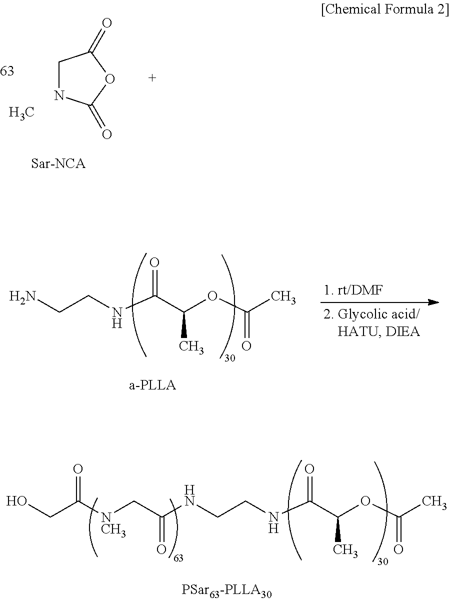

### (PSar66PLLA31)

A linear amphiphilic block polymer (PSar66-PLLA31) comprising a hydrophilic block having 66 sarcosine units and a hydrophobic block having 31 L-lactic acid units was synchesized by the same reaction.

### (Hydrophobic Polymer A2)

The hydrophobic polymer A2 can be synthesized with reference to, for example, WO 2009/148121 A ([0235] to [0243]).

### (PLA0020)

Poly-DL-lactic acid (LA/GA=100/0, weight average molecular weight MW=20,000) was synthesized by ring-opening polymerization of DL-lactide. Here, LA represents a DL-lactic acid unit (racemic mixture of L-lactic acid unit and D-lactic acid unit), GA represents glycolic acid, and LA/GA represents a molar ratio between DL-lactic acid unit and glycolic acid unit. The same applies hereinafter.

The number of DL-lactic acid units in PLA0020:278.

### (PLGA5005)

A DL-lactic acid/glycolic acid copolymer (LA/GA=50/50, weight average molecular weight MW=5,000) was synthesized by ring-opening polymerization of DL-lactide and glycolide. The number of DL-lactic acid units+glycolic acid units in PLGA5005: 77.

### (PLGA5010)

A DL-lactic acid/glycolic acid copolymer (LA/GA=50/50, weight average molecular weight MW=10,000) was synthesized by ring-opening polymerization of DL-lactide and glycolide. The number of DL-lactic acid units+glycolic acid units in PLGA5010: 154.

### (Z-PLLA30: Comparative)

Poly-L-lactic acid (average polymerization degree: 30, weight average molecular weight MW=2,356) represented as Z-PLLA was synthesized using L-lactide (compound 1) and N-carbobentoxy-1,2-diaminoethane hydrochloride (compound 2). The number of L-lactic acid units in Z-PLLA: 30.

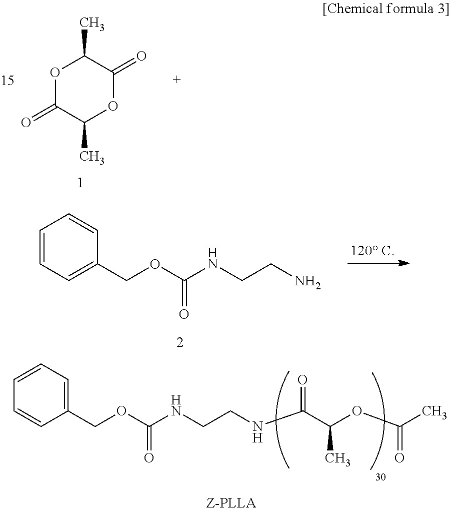

### (DSC Measurement)

Each of the hydrophobic polymers PLA0020, PLGA5005, PLGA5010, and Z-PLLA was analysed by a differential scanning calorimeter (DSC) in the following manner.

About 2 mg of a sample was weighed and placed in a standard aluminum sample container (alumina crimp cell), the sample container was covered with a lid, and the lid was crimped by a sealer crimper (SSC30) to hermetically seal the sample container. As a reference substance, alumina was used. Measurement was performed using DSC-60 (manufactured by SHIMADZO CORPORATION).

The temperature of the sample was increased from 30° C. to 150° C. at a temperature rise rate of 10° C./min. Then, the sample was rapidly cooled from 150° C. to 30° C. In the case of Z-PLLA, a crystallization temperature (about 90° C.) as an exothermic peak and a melting temperature (about 130° C.) as an endothermic peak were observed. Z-PLLA was a crystalline polymer. On the other hand, PLA0020, PLGA5005, and PLGA5010 were all oily, and neither an endothermic reaction nor an exothermic reaction was observed. These polymers were all amorphous.

### Example 1

The amphiphilic polymer A1 (PSar63-PLLA30, or PSar66-PLLA315) and the hydrophobic polymer A2 (PLA0020, PLGA5005, or PLGA5010) were added to a test tube in amounts shown in Table 1 and dissolved in 1 mL of chloroform. The solvent was removed by evaporation under reduced pressure using an evaporator to form a film on the inner wall of the test tube. The evaporation under reduced pressure was performed for 45 minutes with a water bath at 40° C. Further, vacuum drying was performed (room temperature, 5 to 15 Pa, 2 hr), and then 2 mL of distilled water was added and warmed at 85° C. for 20 minutes to form particles. After the particles were formed, the solution was cooled until cooled to room temperature. In this way, A1/A2 lactosome nanoparticles of No. 2 to No. 6 were obtained. Further, lactosome nanoparticles of No. 1 were prepared for reference in the same manner except that the hydrophobic polymer A2 was not used.

The particle diameters of the lactosome nanoparticles No. 1 to No. 6 were measured by dynamic light scattering (DLS). The measurement was performed using a dynamic light scattering measuring instrument (manufactured by Malvern Instruments, Zetasizer Nano).

TABLE 1

| | | | | Addition | | Addition | |
|---|---|---|---|---|---|---|---|
| | | Particle | | amount of | | amount of | Molar |
| | | diameter | Composition | A1 | Composition | A2 | ratio |
| No. | TU_{A2}/TU_{A1} | (nm) | of A1 | (mg) | of A2 | (mg) | A2/A1 |
| 1 | 0 | 34.5 | PSar63-PLLA30 | 2 | — | — | — |
| 2 | 0.64 | 40.4 | PSar63-PLLA30 | 2 | PLGA5005 | 0.38 | 25 |
| 3 | 1.28 | 53.4 | PSar63-PLLA30 | 2 | PLGA5005 | 0.75 | 50 |
| 4 | 2.57 | 131 | PSar63-PLLA30 | 2 | PLGA5005 | 1.51 | 100 |
| 5 | 5.13 | 233 | PSar66-PLLA31 | 2 | PLGA5010 | 2.82 | 100 |
| 6 | 9.27 | 300 | PSar66-PLLA31 | 2 | PLA0020 | 5.65 | 100 |

In Table 1, TU_{A2}/TU_{A1}. represents the ratio of the total number of lactic acid units and glycolic acid units (TU_{A2}) contained in all the hydrophobic polymers A2 constituting the lactos ome nanoparticle to the total number of lactic acid units (TU_{A1}) contained in ail the amphiphilic block polymers A1.

### Comparative Example 1

The amphiphilic polymer A1 (PSar63-PLLA30) and the hydrophobic polymer A2 (Z-PLLA) were added to a test tube in amounts shown in Table 2 and dissolved in 1 mL of chloroform. The solvent was removed by evaporation under reduced pressure using an evaporator to form a film on the inner wall of the test tube. The evaporation under reduced pressure was performed for 45 minutes with a water bath at 40° C. Further, vacuum drying was performed (room temperature, 5 to 15 Pa, 2 hr), and then 2 mL of distilled water was added and warmed at 85° C. for 15 minutes to form particles. After the particles were formed, the solution was cooled until cooled to room temperature. In this way, lactosome nanoparticles of No. 12 to No. 16 were obtained. Further, lactosome nanoparticles of No. 11 were prepared for reference In the same manner except that the hydrophobic polymer A2 was not used. The particle diameters of the lactosome nanoparticles of No. 11 to No. 16 were measured in the same manner as in Example 1.

TABLE 2

| | | | | Addition | | Addition | |
|---|---|---|---|---|---|---|---|
| | | Particle | | amount of | | amount of | Molar |
| | | diameter | Composition | A1 | Composition | A2 | ratio |
| No. | TU_{A2}/TU_{A1} | (nm) | of A1 | (mg) | of A2 | (mg) | A2/A1 |
| 11 | 0 | 30.7 | PSar63-PLLA30 | 2 | — | — | — |
| 12 | 0.5 | 40.5 | PSar63-PLLA30 | 2 | Z-PLLA30 | 0.35 | 50 |
| 13 | 1 | 52.3 | PSar63-PLLA30 | 2 | Z-PLLA30 | 0.71 | 100 |
| 14 | 3 | 84.5 | PSar63-PLLA30 | 2 | Z-PLLA30 | 2.13 | 300 |
| 15 | 5 | 122 | PSar63-PLLA30 | 2 | Z-PLLA30 | 3.55 | 500 |
| 16 | 7 | 122 | PSar63-PLLA30 | 2 | Z-PLLA30 | 4.97 | 700 |

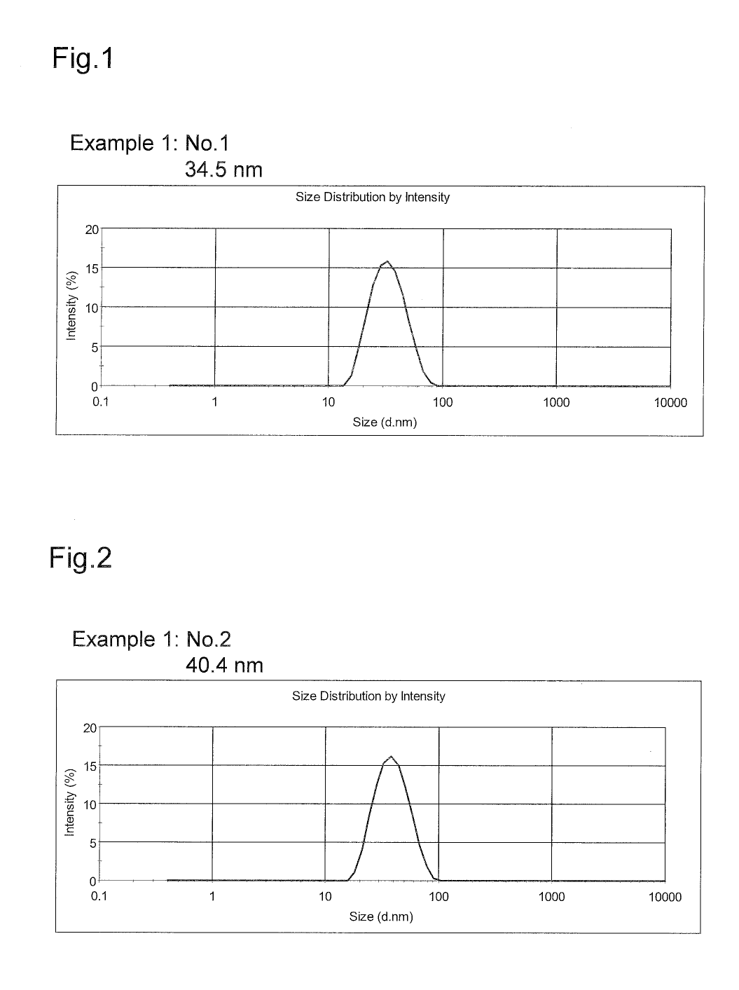

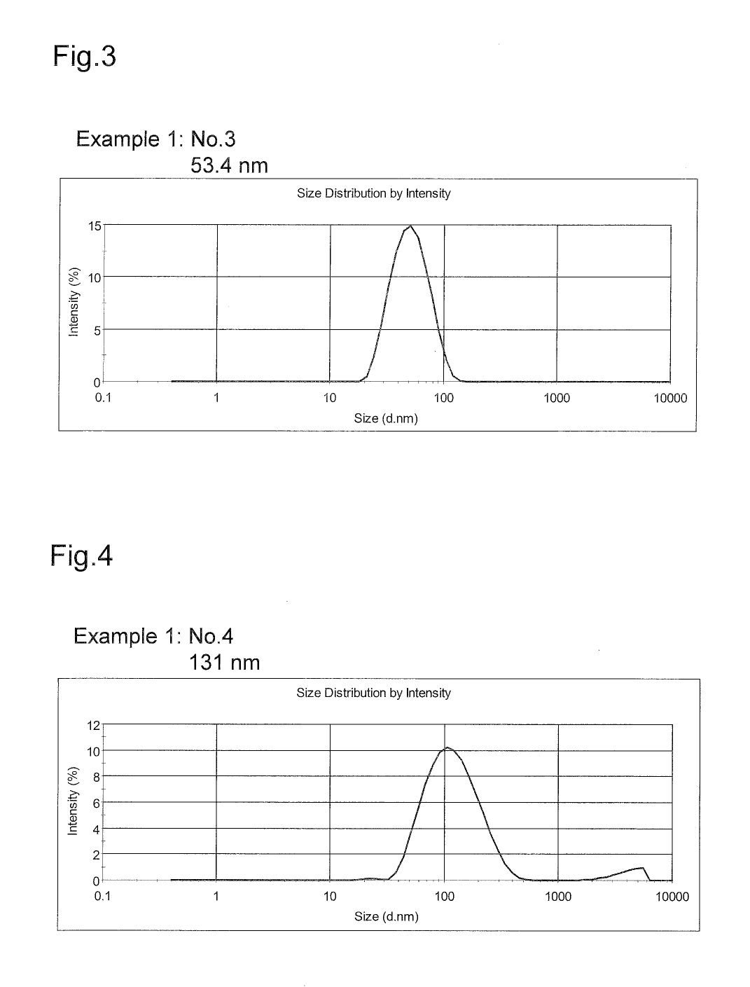

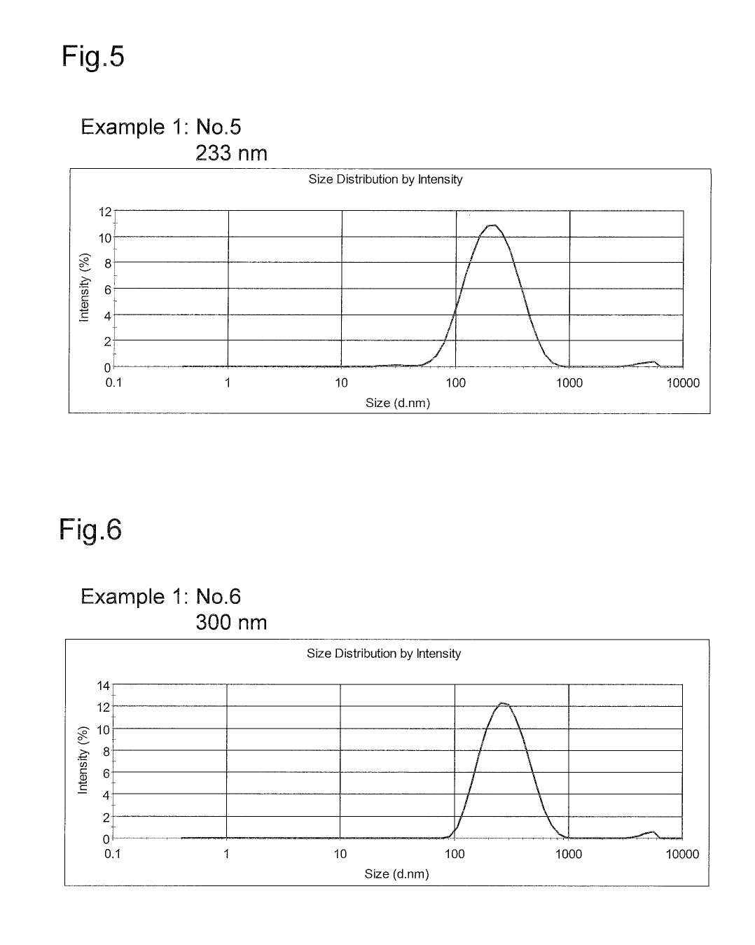

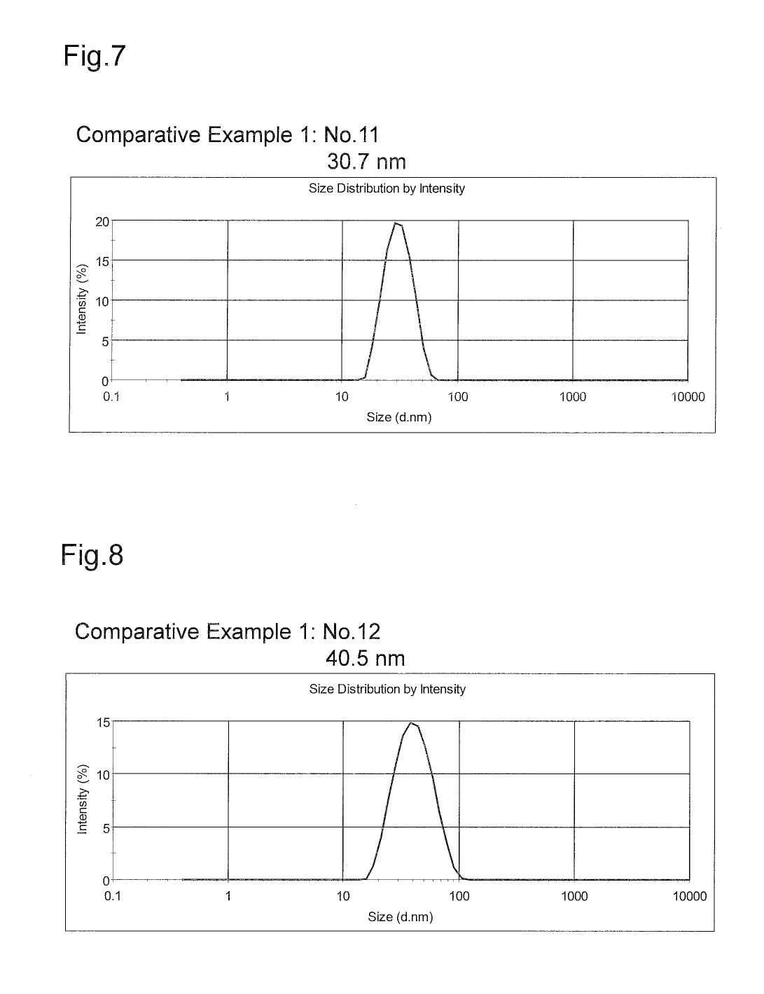

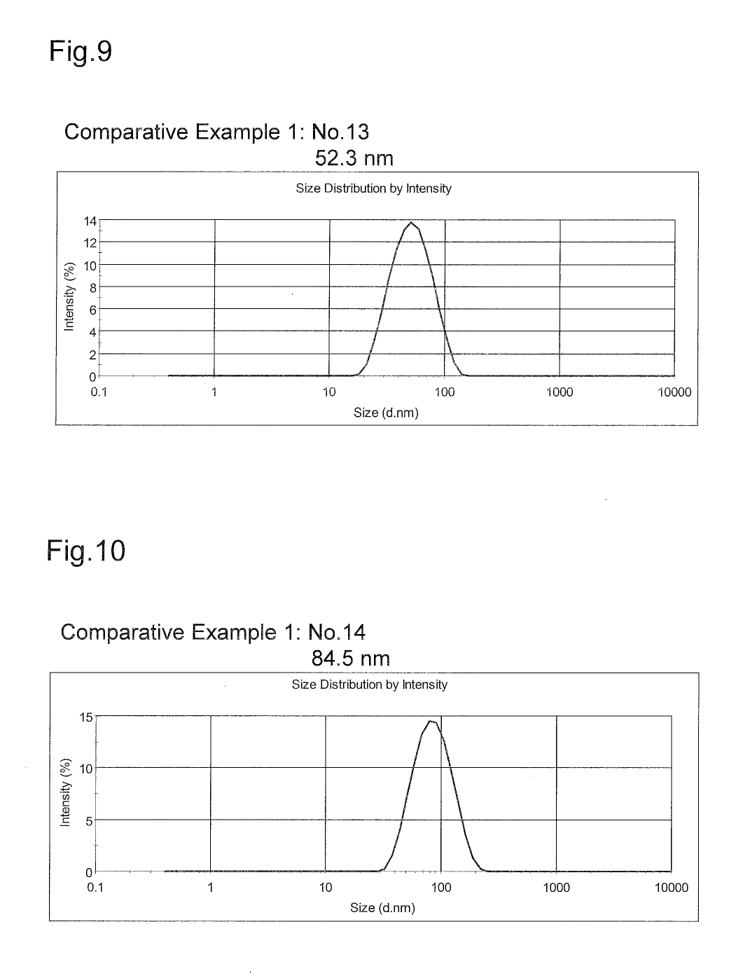

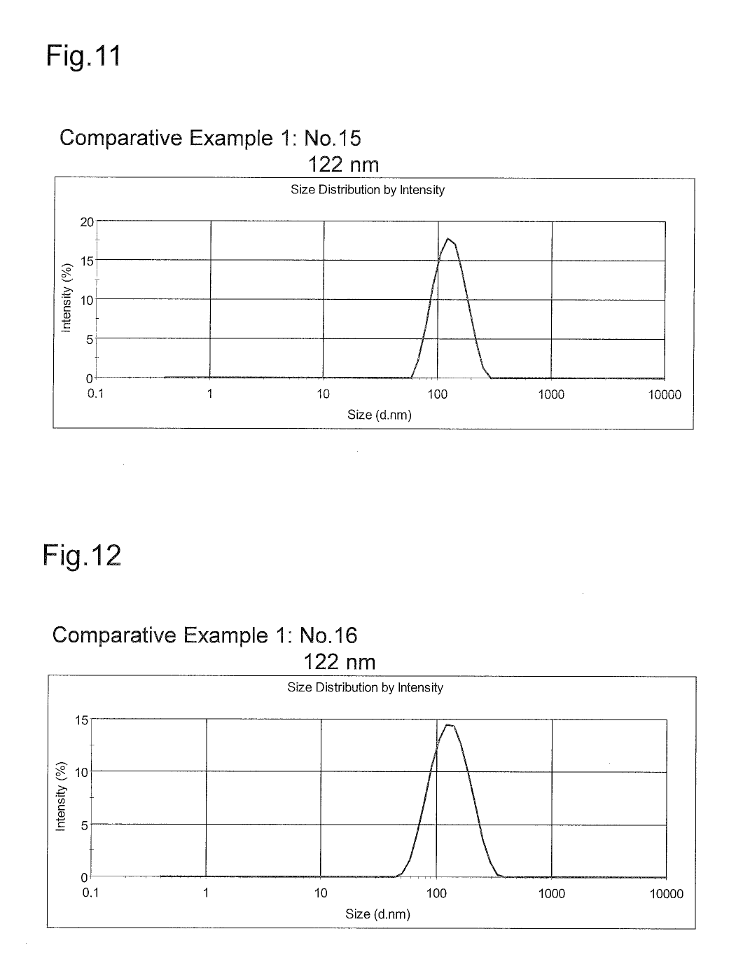

The DLS measurement results of the lactosome nanoparticles (particle size distribution; Size Distribution by Intensity) in Example 1 are shown in FIG. 1 to 6. The DLS measurement results of the lactosome nanoparticles (particle size distribution) in Comparative Example 1 are shown in FIGS. 7 to 12. The horizontal axis represents a particle diameter (sized) (nm) and the vertical axis represents intensity (%).

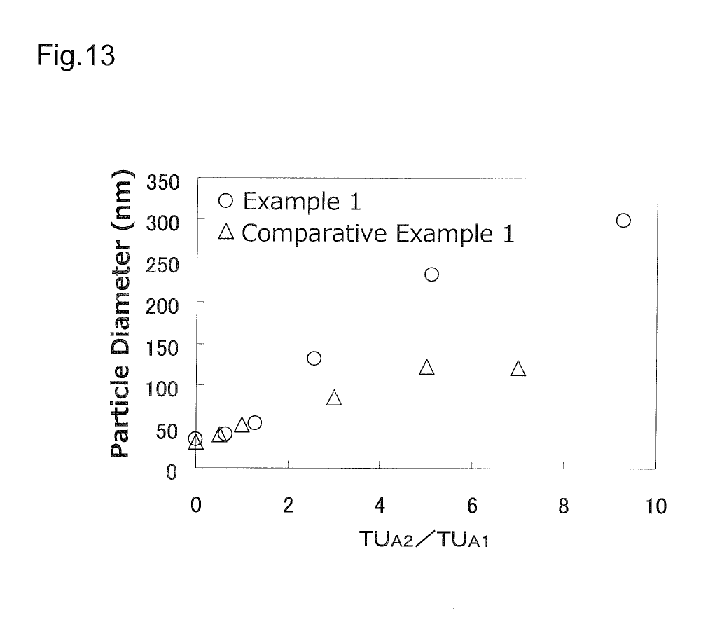

FIG. 13 is a graph showing a comparison between a change in the particle diameter of the lactosome nanoparticles of Example 1 and a change in the particle diameter of the lactosome nanoparticles of Comparative Example 1. In FIG. 13, the horizontal axis represents the ratio (TU_{A2}/TU_{A1}) of the total number of lactic acid units and glycolic acid unite (TU_{A1}) contained in all the hydrophobic polymers A2 constituting the lactosome nanoparticle to the total number of lactic acid units (TU_{A1}) contained in all the amphiphilic block polymers A1, and the vertical axis represents a particle diameter (nm).

As can be seen from FIG. 13, in Example 1, the particle diameter could be continuously controlled from 34.5 nm to 300 nm by changing the ratio (TU_{A2}/TU_{A1}) of the total, number of lactic acid units and glycolic acid units (TU_{A2}) in the hydrophobic polymer A2 to the total number of lactic acid units (TU_{A1}) in the amphiphilic block polymer A1 In the range of 0 to 9.27. On the other hand, in Comparative Example 1, although the ratio was changed in the range of 0 to 7, the particle diameter was changed only from 30.7 nm to 122 nm.

1-11. (canceled)

12. A method for producing a molecular assembly comprising:
an amphophilic block polymer A1 comprising a hydrophilic block having a sarcosine unit and a hydrophobic block having a lactic acid unit; and
an amorphous hydrophobic polymer A2 having an aliphatic hydroxy acid unit, the method comprising;
determining a number of aliphatic hydroxy acid units contained in the amorphous hydrophobic polymer A2 in accordance with a predetermined particle diameter of the molecular assembly,
preparing a solution, in a container, containing the amphiphilic block polymer A1 and the amorphous hydrophobic polymer A2 containing the determined number of aliphatic hydroxy acid units in an organic solvent;
removing the organic solvent from the solution to obtain a film comprising the amphiphilic block polymer A1 and the amorphous hydrophobic polymer A2 on an inner wall of the container; and
adding water or an aqueous solution into the container to convert the film into a particulate molecular assembly, thereby obtaining a dispersion liquid of the molecular assembly.

13. The method for producing a molecular assembly according to claim 12, wherein the number of aliphatic hydroxy acid units contained in the amorphous hydrophobic polymer A2 exceeds twice a number of lactic acid units contained in the hydrophobic block of the amphiphilic block polymer A1.

14. The method for producing a molecular assembly according to claim 12, wherein the hydrophilic block contains 2 to 300 sarcosine units.

15. The method for producing a molecular assembly according to claim 12, wherein the hydrophobic block contains 5 to 400 lactic acid units.

16. The method for producing a molecular assembly according to claim 12, wherein the amorphous hydrophobic polymer A2 has, as the aliphatic hydroxy acid unit, at least one selected from the group consisting of a lactic acid unit and a glycolic acid unit.

17. The method for producing a molecular assembly according to claim 12, wherein the amorphous hydrophobic polymer A2 contains 35 or more aliphatic hydroxy acid units.

18. The method for producing a molecular assembly according to claim 12, wherein the amorphous hydrophobic polymer A2 contains 200 or more aliphatic hydroxy acid units.

19. The method for producing a molecular assembly according to claim 12, wherein a molar ratio A2/A1 of the amorphous hydrophobic polymer A2 to the amphiphilic block polymer A1is in the range of 0.1/1 to 10/1.

20. The method for producing a molecular assembly according to claim 12, wherein the molecular assembly has a particle diameter of 10 to 1,000 nm.

21. A method for producing a molecular assembly comprising;
an amphiphilic block polymer A1 comprising a hydrophilic block having a sarcosine unit and a hydrophobic block having a lactic acid unit; and
an amorphous hydrophobic polymer A2 having an aliphatic hydroxy acid unit, the method comprising:
determining a number of aliphatic hydroxy acid units contained in the amorphous hydrophobic polymer A2 in accordance with a predetermined particle diameter of the molecular assembly;
preparing a solution, in a container, containing the amphiphilic block polymer A1 and the amorphous hydrophobic polymer A2 containing the determined number of aliphatic hydroxy acid units in an organic solvent;
dispersing the solution into water or an aqueous solution; and
removing the organic solvent.

22. The method for producing a molecular assembly according to claim 21, wherein the number of aliphatic hydroxy acid units contained in the amorphous hydrophobic polymer A2 exceeds twice a number of lactic acid units contained in the hydrophobic block of the amphiphilic block polymer A1.

23. The method for producing a molecular assembly according to claim 21, wherein the hydrophilic block contains 2 to 300 sarcosine units.

24. The method for producing a molecular assembly according to claim 21, wherein the hydrophobic block contains 5 to 400 lactic acid units.

25. The method for producing a molecular assembly according to claim 21, wherein the amorphous hydrophobic polymer A2 has, as the aliphatic hydroxy acid unit, at least one selected from the group consisting of a lactic acid, unit and a glycolic acid unit.

26. The method for producing a molecular assembly according to claim 21, wherein the amorphous hydrophobic polymer A2 contains 35 or more aliphatic hydroxy acid units.

27. The method for producing a molecular assembly according to claim 21, wherein, the amorphous hydrophobic polymer A2 contains 200 or more aliphatic hydroxy acid units.

28. The method for producing a molecular assembly according to claim 21, wherein a molar ratio A2/A1 of the amorphous hydrophobic polymer A2 to the amphiphilic block polymer A1 is in the range of 0.1/1 to 10/1.

29. The method for producing a molecular assembly according to claim 21, wherein the molecular assembly has a particle diameter of 10 to 1,000 nm.
